(19) United States
(12) Patent Application Publication
(10) Pub. No.: US 2007/0231397 A1
(43) Pub. Date: Oct. 4, 2007

(54) Method for Producing Coated Drugs Having a Stable Profile for the Release of Active Ingredients

(75) Inventors: Hans-Ulrich Petereit, Darmstadt (DE); Wolfgang Weisbrod, Darmstadt (DE); Hans Bar, Brombachtal (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: ROEHM GMBH, Darmstadt (DE)

(21) Appl. No.: 11/570,050

(22) PCT Filed: Jul. 9, 2005
(86) PCT No.: PCT/EP05/07468

§ 371 Date: Dec. 5, 2006

(30) Foreign Application Priority Data
Jul. 23, 2004 (DE) 10 2004 035 938.5

(51) Int. Cl. A61K 9/00

(52) U.S. Cl. 424/490

(57) ABSTRACT

The invention relates to methods for producing drugs having a stable profile for the release of active ingredients, said drugs exhibiting a controlled release characteristic as a result of the coating of vinyl (co)polymers. The inventive methods are characterised in that the coated drugs are conditioned in a fluidised bed coating appliance or a drum coating appliance for at least 10 minutes until a stable profile for the release of active ingredients is reached at a temperature of between 30 and 70° C. A humidity of between 5 and 30% is regulated during the conditioning process.

The invention relates to a method for producing pharmaceutical forms having a stable active ingredient release profile, the pharmaceutical forms having controlled release characteristics on account of a coating of vinyl (co)polymers.

### PRIOR ART

U.S. Pat. No. 4,600,645 describes a process for producing coated dosage forms having a stable active ingredient release profile. For this, a drug-containing substrate should be coated with a polymer delaying the release of active ingredients and immediately thereafter be provided with a water-soluble second coating. The dosage form obtained should then be dried for 15 to 60 minutes. In one example, drug-containing pellets are first coated with ethylcellulose (Aquacoat®) and subsequently with hydroxypropylmethylcellulose (HPMC). Drying is carried out directly in a fluidized bed apparatus used for coating, e.g. at 70° C. for 30 minutes and at a rotor speed of 100 rpm. Coated drug-containing pellets which have been dried at 58° C. for 60 minutes have approximately the same release profile as in the case of drying for 60° C. for one week in a heated cabinet.

EP 0 553 392 A1 describes pharmaceutical forms having acrylic polymer coatings having a stabilized active ingredient release profile prepared from aqueous dispersions. The coatings can consist, for example, of polymers of the type EUDRAGIT® RL/RS. A stable active ingredient release profile is achieved by conditioning the coated pharmaceutical forms in a heated cabinet at a temperature around 450° C. for 24 to 48 hours.

U.S. 5,273,760 describes pharmaceutical forms coated with ethylcellulose from aqueous dispersion having a stabilized active ingredient release profile. Here, by storage for a number of days e.g. for 72 hours, at 60° C. and 60-80% atmospheric humidity approximately the same effect is achieved as with storage for one month at 37° C. and 80% atmospheric humidity.

### OBJECT AND ACHIEVEMENT

Pharmaceutical forms which display controlled release characteristics on account of a coating of vinyl (co)polymers do not yet exhibit a reproducible release profile immediately after the application of the spray suspensions. According to experience, this effect is most pronounced in the case of coatings which have been prepared from aqueous dispersions, but also comes to bear in the case of coatings which have been applied from organic solutions. Often, the manufacturers of appropriate vinyl (co)polymers recommend to their customers who prepare pharmaceutical forms therewith to add a longer drying phase of, for example, 16 to 48 hours after the application of the coatings in order to obtain stable profiles for the release of active ingredient. This procedure has proven itself in practice, but is laborious, in particular with large batches of pharmaceutical.

EP 0 553 392 A1 proposes, similarly to the above-mentioned recommendations of the manufacturers, conditioning in a heated cabinet at a temperature around 45° C. for 24 to 48 hours for the achievement of a reproducible profile for the release of pharmaceutical form using acrylic polymer coatings prepared from aqueous dispersions. This procedure has proven itself in practice, but is laborious, in particular with large batches of pharmaceutical.

U.S. Pat. No. 4,600,645 describes a conditioning method in which the pharmaceutical forms should be dried for 15 to 60 minutes. The inventors have found, however, that this method is obviously suitable only for coatings based on cellulose and not for pharmaceutical forms coated with vinyl (co)polymers. Coatings of vinyl (co)polymers can easily become cracked in the case of conditioning according to U.S. Pat. No. 4,600,645. The cracks, which are visible under the microscope, lead to an uncontrolled release of active ingredient and prevent the achievement of a stabilized state from the start.

It was seen as an object to provide a method for pharmaceutical forms which, on account of a coating of vinyl (co)polymers, have controlled release characteristics, which makes it possible in a short time and with little expenditure to arrive at stable active ingredient release profiles.

The object is achieved by a

method for producing pharmaceutical forms having a stable active ingredient release profile, the pharmaceutical forms having controlled release characteristics on account of a coating of vinyl (co) polymers,

characterized in that
   the coated pharmaceutical forms are conditioned at a temperature of 30 to 70° C. in a fluidized bed coating apparatus or a drum coating apparatus for at least 10 minutes until achievement of a stable active ingredient release profile, an atmospheric humidity of 5 to 30% being set during the conditioning.
   Achievement of the Invention

The invention relates to a method for producing pharmaceutical forms which, on account of a coating of vinyl (co)polymers, have controlled release characteristics, with a stable release profile.

Controlled Release Characteristics

The term “controlled” release characteristics is known to the person skilled in the art. The person skilled in the art understands by this, for example, that the release profile for certain active ingredients can be tailored reproducibly to the active ingredient by means of the formulation of the pharmaceutical form, in particular by the choice of the external coating. Known “controlled ” release characteristics are the pH-controlled release of active ingredient or the delayed, also designated as retarded, release of active ingredient. After the release of the pharmaceutical form for sale, the release profile must no longer significantly change even after relatively long storage, in order to guarantee the therapeutic action in vivo. Standardized methods for the determination of the release profile are adequately known to the person skilled in the art. USP27-NF22Supplement 1 <711> Dissolution and <724> Drug Release describe methods for the determination of the release profile and permissible tolerances.

Coatings of vinyl (co)polymers which impart controlled release cnaracteristics to pharmaceutical forms are adequately known. Mention may be made, in particular, of coatings of vinyl (co)polymers of the taupe consisting of the (meth)acrylate (co)polymers or of the type consisting of the polyvinyl acetates including the derivatives of polyvinyl acetate.

As a result of their pH-independent, slowly swelling character in intestinal juice, coatings of methacrylate copolymers having neutral radicals, methacrylate copolymers having functional quaternary amino groups, and polyvinyl acetates cause a retarding, controlled release of active ingredient. As a result of the alkaline medium of the intestine, coatings of meth-acrylate copolymers having anionic functional groups cause pH-controlled, controlled release of active ingredient. Coatings of methacrylate copolymers having functional tertiary amino groups serve for taste isolation and owing to the acidic medium of the stomach cause pH-controlled, rapid controlled release of active ingredient.

The process is in particular suitable for pharmaceutical forms having coatings of vinyl (co)polymers, which have been applied from aqueous dispersions. The process is furthermore suitable, in particular, for pharmaceutical forms having coatings which cause a retarding release of active ingredient.

Stable Active Ingredient Release Profile

Within the meaning of USP27-NF22 Supplement 1 <711> Dissolution and <724> Drug Release, deviation of more than +/−10% of the declared amount of active ingredient for the respective pharmaceutical form or individual dose can be regarded as a significant, non-tolerable change, which can lead in vivo to a modified therapeutic action. In this case, the pharmaceutical form could be described as unstable. The term “stable active ingredient release profile” is therefore defined as a person skilled in the art understands it taking into consideration USP-NF.

A stable active ingredient release profile within the meaning of the invention is understood as meaning an active ingredient release profile which, compared to an active ingredient release profile of a reference preparation which has been conditioned in a recirculating air drying cabinet for 24 hours at 40° C., differs by not more than +/−10%. The % data relate here to the declared active ingredient content of the individual dose for the respective pharmaceutical form at the licensing authority (e.g. EMEA in Europe or FDA in the USA) and the declared measurement times.

For example, for a pharmaceutical form “X” in tablet form, the amount of active ingredient “Y” can be declared for a tablet. At the times “Z₁” and “Z₂” and “Z₃”, in each case specified aliquot amounts “T₁”, “T₂” and “T₃” of the amount of active ingredient “Y” must then be released under defined conditions. Permissible deviations at the times indicated, starting from the respective theoretical value of the indicated aliquot amounts “T₁”, “T₂” and “T₃” are not more than +/−10% of the amount of active ingredient “Y”. If, for example, the value “T₂” is 50%, values in the range from 40 to 60% are to be regarded as stable.

The reference value corresponds here to the method proposed in EP 0 553 392 A1, with which stable release profiles are achieved in pharmaceutical forms which have controlled release characteristics on account of a coating of certain vinyl (co)polymers. The inventors have found that the method of EP 0 553 392 A1 is generally suitable as a reference value for vinyl (co)polymers having controlled release characteristics.

As an active ingredient release curve or release characteristics, the active ingredient release curve or release characteristics demanded or relevant for the particular pharmaceutical form with respect to required therapeutic action is to be used as a basis here. This is different virtually for every pharmaceutical form, depending on the active ingredient contained and the polymer coating type.

The active ingredient release curve of a pharmaceutical form having a retarding polymer coating type can be recorded, for example, for 4 to 12 hours at a constant pH. The active ingredient release curve of a pharmaceutical form having an anionic polymer coating type is as a rule only recorded for 2 hours at pH 1.2, for the gastric juice resistance test, and subsequently for a number of hours at a constant higher pH, for the active ingredient release test. The release curve of a pharmaceutical form having a cationic polymer coating type containing tertiary amino groups can be recorded, for example, at a constant pH over a short time of 10 to 60 at least, since these coating types dissolve comparatively rapidly.

The Preparation of Pharmaceutical Forms Coated with Vinyl (co)Polymers

In a manner known per se, active ingredient-containing cores or pellet cores form the basis for the coatings of vinyl (co)polymers. pelletizing can be carried out on active ingredient-free spheres (nonpareils) or core-free pellets, pellet cores, can be produced. First, a rounded, active ingredient-containing substrate with or without a core is produced. By means of a fluidized bed process, liquid can be applied to placebo pellets or other suitable carrier materials, the solvent or suspending agent being evaporated. According to the preparation process, a drying step can be added. The still uncoated, rounded layer is designated, for example, as a core or pellet core, according to size.

The active ingredient is as a rule brought into an organic solvent or into water and mixed. In order to guarantee the satisfactory sprayability of the mixture, it is usually necessary to formulate a mixture with relatively low viscosity. The addition of a detergent, e.g. Tween in concentrations of 0.1 to 20, preferably 0.5 to 10% by weight, can be advantageous for the reduction of the surface tension. In addition to the active ingredient, they can contain further pharmaceutical excipients: binders, such as cellulose and its derivatives, polyvinylpyrrolidone (PVP), moisture retention agents, disintegration promoters, lubricants, disintegrants, (meth)acrylates, starch and its derivatives, sugar solubilizers or others.

Appropriate application processes are known, for example, from Bauer, Lehmann, Osterwald, Rothgang “Überzogene Arzneiformen” [Coated Pharmaceutical Forms] Wissenschaftliche Verlagsgesellschaft mbH Stuttgart, Chap. 7, pp. 165-196.

Details are furthermore known to the person skilled in the art from textbooks. See, for example:
Voigt, R. (1984): Lehrbuch der pharmazeutischen Technologie [Textbook of Pharmaceutical Technology]; Verlag Chemie Weinheim—Deerfield Beach/Florida—Basle.
Sucker, H. Fuchs, P., Speiser, P.: Pharmazeutische Technologie [Pharmaceutical Technology], George Thieme Verlag Stuttgart (1991), in particular chapters 15 and 16, pp. 626 -642.
Gennaro, A., R. (Editor), Remington's Pharmaceutical Sciences, Mack Publishing Co., Easton Pennsylvania (1985), Chapter 88, pp. 1567-1573.
List, P. H. (1982): Arzneiformenlehre [Pharmaceutical Form Theory], Wissenschaftliche Verlagsgesellschaft mbH, Stuttgart.

Pellet cores can be rounded by processes such as rotagglomeration, precipitation or spray processes, in particular ultrasonic vortex spray processes, to give still uncoated cores or pellet cores of defined size, e.g. 50 to 1000 μm. This has the advantage that the entire core volume is available for active ingredient loading. The active ingredient loading can thereby again be increased in relation to the embodiment having an inert core.

After preparation of the active ingredient-containing cores or pellet cores these are provided in spray processes with an outer coating of vinyl (co)polymers, such that coated pellets are obtained. The pellets are prepared by means of spray application from organic solution, or preferably from aqueous dispersions. For implementation, it is crucial here that uniform, pore-free coatings result. As a rule, the coated pellets are additionally subsequently dried for a few minutes after the spray application before the conditioning process is begun. As a rule, the polymer coatings contain pharmaceutically customary excipients such as, for example, release agents or plasticizers.

The Conditioning Process

The coated pharmaceutical forms are conditioned in a fluidized bed coating apparatus (fluidized bed coater) or a drum coating apparatus (drum coater) for at least 10 minutes until a stable active ingredient release profile is achieved. This condition is as a rule fulfilled with a drying time of 10 to 120, 15 to 90, in particular 15 to 60, minutes. The drying temperature is 30 to 70, preferably 35 to 65, particularly preferably 40 to 60° C.

The equipment, the fluidized bed coating apparatus (fluidized bed coater) or the drum coating apparatus (drum coater) are known to the person skilled in the art from galenics. In the fluidized bed coating apparatus, active ingredient-containing cores or active ingredient-containing pellets can be provided, for example, with a coating of (co)polymers. For this purpose, the active ingredient-containing cores or active ingredient-containing pellets are fluidized in a stream of air to give a permanent to and fro movement, while the (meth)acrylate copolymer is simultaneously sprayed in in the form of a finely nebulized dispersion. The polymer dispersion precipitates on the active ingredient-containing cores or active ingredient-containing pellets and forms a film there. The water contained evaporates in the stream of air, in which, as a rule, a temperature in the range from 30 to 70° C. is set. Drum coating apparatus (drum coater) the movement of the active ingredient-containing cores or active ingredient-containing pellets takes place by means of the movement of the drum.

If conditioning is carried out for significantly longer than necessary, when the unit, for example, is allowed to run overnight, adverse mechanical effects can occur as a result of abrasion, whereby the initially achieved stable active ingredient release profile is again lost. Drying is therefore carried out only until the stable active ingredient release profile is achieved, or until in the region of a stable active ingredient release profile, but not, or not significantly, beyond it.

During the drying, an atmospheric humidity of 5 to 30 preferably 10 to 25% is set. The atmospheric humidity relates to the respective drying temperature and can be measured or determined using appropriate sensors downstream of the filter unit in the waste air of the fluidized bed coating apparatus, for example. The necessary atmospheric humidity can expediently be produced by spraying in waters.

To avoid tackiness and to increase the mechanical strength, advantageously an aqueous suspension comprising 0.1 to 5, preferably 0.2 to 1% by weight of a release agent, e.g. talc, magnesium stearate or a silicic acid (e.g. of Syloid® type), based on the suspension, can be sprayed in.

The process can preferably be carried out very efficiently if the coated pharmaceutical forms are prepared in a first step coated pharmaceutical forms by coating active ingredient-containing cores or pellets with (meth)acrylate copolymers in the fluidized bed coating apparatus or a drum coating apparatus and in a second step carrying out the conditioning of the coated pharmaceutical forms in the same apparatus immediately after the preparation.

The conditioned pellets obtained can preferably be further processed by means of pharmaceutically customary excipients and in a manner known per se to give a multiparticulate pharmaceutical form, in particular to give pellet-containing tablets, mini-tablets, capsules, sachets or inspissated juices.

Coatings of Neutral Vinyl (Co)Polymers

a) Neutral (Meth)Acrylate Copolymers, EUDRAGIT® NE Type

The process according to the invention is suitable for pharmaceutical forms whose coatings consist of (meth)acrylate (co)polymers which are polymerized to more than 95% by weight to 100% from monomers containing neutral radicals. Monomers containing neutral radicals can in particular be C₁- to C₄-alkyl esters of acrylic or methacrylic acid are in particular methyl methacrylate, ethyl methacrylate, butyl methacrylate, methyl acrylate, ethyl acrylate and butyl acrylate.

Mention may be made, for example, of neutral (meth)acrylate copolymers consisting of 20 to 40% by weight of ethyl acrylate and 60 to 80% by weight of methyl methacrylate (EUDRAGIT® NE type). Optionally, the largely neutral (meth)acrylate copolymers mentioned can contain small proportions, e.g. 0 less than 5, preferably 0 to 2% by weight, of (meth)acrylate monomers having an anionic group in the alkyl radical can, for example acrylic acid, but preferably methacrylic acid. The mainly or completely neutral copolymer preferably has the property of swelling above pH 5.0 in water or in the intestinal juice medium and releasing active ingredient.

EUDRAGIT® NE is a copolymer of 30% by weight of ethyl acrylate and 70% by weight of methyl methacrylate. The polymer can be employed, for example, in the form of a 30% strength aqueous dispersion, EUDRAGIT® NE 30D.

b) Polyvinyl Acetates

The inner coating can contain a (co)polymer which is polyvinyl acetate or a polyvinyl acetate. The term “a polyvinyl acetate” additionally includes derivatives of polyvinyl acetate. The polyvinyl acetate can be present as a dispersion (e.g. of the type Kollicoat® SR 30 D, manufacturer BASF, polyvinyl acetate dispersion, stabilized with povidone and Na laurylsulfate).

Methacrylate copolymers containing functional anionic radicals

The process is suitable for pharmaceutical forms having coatings of (meth)acrylate copolymers which consist to 25 to 95% by weight of free radical-polymerized C₁- to C₄-alkyl esters of acrylic or methacrylic acid and to 5 to 75% by weight of (meth)acrylate monomers containing an anionic group in the alkyl radical.

As a rule, the proportions mentioned add up to 100% by weight. However, without this leading to an adverse effect on or change in the essential properties, small amounts in the range from 0 to 10, e.g. 1 to 5% by weight, of further vinylically copolymerizable monomers, such as, for example, hydroxyethyl methacrylate or hydroxyethyl acrylate, can additionally be present.

C₁- to C₄-alkyl esters of acrylic or methacrylic acid are, in particular, methyl methacrylate, ethyl methacrylate, butyl methacrylate, methyl acrylate, ethyl acrylate and butyl acrylate.

A (meth)acrylate monomer containing an anionic group in the alkyl radical can be, for example, acrylic acid, but preferably methacrylic acid.

EUDRAGIT® L, L100-55, S and FS Coating Types

The process is suitable for pharmaceutical forms having coatings of (meth)acrylate copolymers consisting of 40 to 60% by weight of methacrylic acid and 60 to 40% by weight of methyl methacrylate or 60 to 40% by weight of ethyl acrylate (EUDRAGIT® L or EUDRAGIT® L100-55 types).

EUDRAGIT® L is a copolymer of 50% by weight of methyl methacrylate and 50% by weight of methacrylic acid. EUDRAGIT® L 30D is a dispersion comprising 30% by weight of EUDRAGIT® L.

EUDRAGIT® L100-55 is a copolymer of 50% by weight of ethyl acrylate and 50% by weight of methacrylic acid. EUDRAGIT® L 30-55 is a dispersion comprising 30% by weight of EUDRAGIT® L 100-55.

The process is suitable for pharmaceutical forms having coatings of (meth)acrylate copolymers consisting of 20 to 40% by weight of methacrylic acid and 80 to 60% by weight of methyl methacrylate (EUDRAGIT® S type).

The process is suitable for pharmaceutical forms having coatings of (meth)acrylate copolymers consisting of 10 to 30% by weight of methyl methacrylate, 50 to 70% by weight of methyl acrylate and 5 to 15% by weight of methacrylic acid (EUDRAGIT® FS type).

EUDRAGIT® FS is a copolymer of 25% by weight of methyl methacrylate, 65% by weight of methyl acrylate and 10% by weight of methacrylic acid. EUDRAGIT® FS 30D is a dispersion comprising 30% by weight of EUDRAGIT® FS.

EUDRAGIT® coating types having a medium content of methacrylic acid

The process is suitable for pharmaceutical forms having coatings of (meth)acrylate copolymers consisting of anionic (meth)acrylate copolymers consisting of 20 to 34% by weight of methacrylic acid and/or acrylic acids 20 to 69% by weight of methyl acrylate and 0 to 40% by weight of ethyl acrylate and optionally 0 to 10% by weight of further vinylically copolymerizable monomers, with the proviso that the glass transition temperature of the copolymer according to ISO 11357-2, item 3.3.3, is at most 60° C. (EUDRAGIT® type having a medium content of methacrylic acid).

The copolymer is in particular composed of free radical-polymerized units of

20 to 34, preferably 25 to 33, particularly preferably 28 to 32% by weight of methacrylic acid or acrylic acid; methacrylic acid is preferred,

20 to 69, preferably 35 to 65, particularly preferably 35 to 55% by weight of methyl acrylate and optionally

0 to 40, preferably 5 to 35, particularly preferably 15 to 35% by weight of ethyl acrylate, with the proviso that the glass transition temperature of the copolymer (without plasticizer addition) according to ISO 11357-2, item 3.3.3, is at most 60, preferably 40 to 60, particularly preferably 45 to 55° C.

The (meth)acrylate copolymer can consist essentially to exclusively of the monomers methacrylic acid, methyl acrylate and ethyl acrylate in the quantitative proportions indicated above. As a rule, the proportions mentioned add up to 100% by weight. However, without this leading to an adverse effect on or change in the essential properties, small amounts in the range from 0 to 10, e.g. 1 to 5% by weight of further vinylically copolymerizable monomers, such as, for example, methyl methacrylate, butyl methacrylate, butyl acrylate or hydroxyethyl methacrylate can additionally be present.

(Meth)acrylate Copolymer Containing Functional Cationic Radicals

a) (Meth)Acrylate Copolymers Containing Tertiary Amino Groups, EUDRAGIT® E100 and EPO Type

The process is suitable for pharmaceutical forms having coatings of (meth)acrylate copolymers of 30 to 80% by weight of free radical-polymerized C₁- to C₄-alkyl esters of acrylic or of methacrylic acid and 70 to 20% by weight of (meth)acrylate monomers containing a tertiary amino group in the alkyl radical.

Suitable monomers containing functional tertiary amino groups are listed in U.S. Pat. No. 4,705,695, column 3, line 64 to column 4, line 13. In particular, mention may be made of dimethylaminoethyl acrylate, 2-dimethylaminopropyl acrylate, dimethylaminopropyl methacrylate, dimethyl-aminobenzyl acryl ate, dimethylaminobenzyl methacrylate, 3-dimethylamino-2,2-dimethyl)propyl acrylate, dimethylamino-2,2-dimethyl)propyl methacrylate, (3-diethylamino-2,2-dimethyl) propyl acrylate and diethyl-amino-2,2-dimethyl) propyl methacrylate. Dimethylamino-ethyl methacrylate is particularly preferred.

The content of monomers containing tertiary amino groups in the copolymer can be between 20 and 70% by weight, preferably between 40 and 60% by weight. The proportions of the C₁- to C₄-alkyl esters of acrylic or methacrylic acid is 70-30% by weight. Mention may be made of methyl methacrylate, ethyl methacrylate, butyl methacrylate, methyl acrylate, ethyl acrylate and butyl acrylate.

A customary (meth)acrylate copolymer containing tertiary amino groups can be synthesized, for example, from 20-30% by weight of methyl methacrylate, 20-30% by weight of butyl methacrylate and 60-40% by weight of dimethylaminoethyl methacrylate.

A commercially customary (meth)acrylate copolymer containing tertiary amino groups is synthesized, for example, from 25% by weight of methyl methacrylate, 25% by weight of butyl methacrylate and 50% by weight of dimethylaminoethyl methacrylate (EUDRAGIT® E100).

A further commercially customary (meth)acrylate copolymer containing tertiary amino groups is, for example, EUDRAGIT® E PO: copolymer of methyl methacrylate, butyl methacrylate, and dimethylamino-ethyl methacrylate in the ratio of 25:25:50 having a mean particle size of 15 μm

b) (Meth)Acrylate Copolymers Containing Quaternary Amino Groups, EUDRAGIT® RS or RL Type

The process is in particular suitable for pharmaceutical forms having coatings of (meth) acrylate copolymers with quaternary ammonium groups, in particular of copolymers which are synthesized from free radical-polymerized units of 50-70% by weight of methyl methacrylate, 20-40% by weight of ethyl acrylate and 12-2% by weight of 2-trimethylammonium ethyl methacrylate chloride EUDRAGIT® RS or RL typed The process is in particular suitable for pharmaceutical forms having coatings of (meth)acrylate copolymers with quaternary ammonium groups which were applied from aqueous dispersions.

The process is in particular suitable for pharmaceutical forms having coatings of (meth)acrylate copolymers which are synthesized from free radical-polymerized units of 65% by weight of methyl methacrylate, 30% by weight of ethyl acrylate and 5% by weight of 2-trimethylammonium ethyl methacrylate chloride (EUDRAGIT® RS).

The process is in particular suitable for pharmaceutical forms having coatings which consist of copolymers which from free radical-polymerized units of 60% by weight of methyl methacrylate, 30% by weight of ethyl acrylate and 10% by weight of 2-trimethyl-ammonium ethyl methacrylate chloride (EUDRAGIT® RL).

The process is in particular suitable for pharmaceutical forms having coatings which consist of mixtures of EUDRAGIT® RS and EUDRAGIT® RL.

Active Ingredients

The process according to the invention is suitable for, in principle, for all pharmaceutical forms coated with vinyl (co)polymers. The pharmaceutical forms can contain, for example, the following active ingredients, active ingredient classes or active ingredient types.

Customary pharmaceuticals can be gathered from reference works, such as, for example, the Rote Liste or the Merck Index.

Biologically Active Substances:

Pharmaceuticals are Intended to be used on or in the human or Animal Body, in Order

1. To cure, to alleviate, to prevent or to recognize diseases, suffering, physical defects or pathological symptoms.
2. To be able to diagnose the condition, the state or the functions of the body or mental states.
3. To replace active ingredients or body fluids produced by the human or animal body.
4. To protect against, to eliminate or to render harmless pathogens, parasites or exogenous substances or
5. To influence the condition, the state or the functions of the body or mental states.
   Therapeutic Classes

These pharmaceutically active substances can belong to one or more active ingredient classes, such as ACE inhibitors, adrenergics, adrenocorticosteroids, acne therapeutics, aldose reductase inhibitors, aldosterone antagonists, alpha-glucosidase inhibitors, alpha 1 antagonists, agents against alcohol abuses, amino acids, amebicides, anabolics, analeptics, anesthetic additives, anesthetics (not inhalative), anesthetics (local), analgesics, androgens, angina therapeutics, antagonists, antiallergics, antiallergics such as PDE inhibitors, antiallergics or asthma treatment, further antiallergics (e.g. leukotriene antagonists, anti-anemics, antiandrogens, antianxiolytics, anti-arthritics, antiarrhythmics, antiatherosclerotics, antibiotics, anticholinergics, anticonvulsives, anti-depressives, antidiabetics, antidiarrheals, anti-diuretics, antidotes, antiemetics, antiepileptics, antifibrinolytics, antiepileptics, antihelmintics, antihistaminics, antihypotensives, antihypertensives, antihypertonics, antihypotonics, anticoagulants, anti-mycotics, antiestrogens, antiestrogens (nonsteroids), antiparkinson agents, antiinflammatories, anti-proliferative active ingredients, antiprotozoal active ingredients, antirheumatics, antischistosomicides, antispasmolytics, antithrombotics, antitussives, appetite suppressants, arteriosclerotic agents, bacteriostatics, beta-blockers, beta receptor blockers, bronchodilators, carboanhydrase inhibitors, chemo-therapeutics, choleretics, cholinergics, cholinergic agonists, cholinesterase inhibitors, agents for the treatment of ulcerative colitis, cyclooxygenase inhibitors, diuretics, ectoparasiticides, emetics, enzymes, enzyme inhibitors, enzyme inhibitors, active ingredients against vomiting, fibrinolytics, fungi-statics, gabapentin gout agents, glaucoma therapeutics, glucocorticoids, glucocorticosteroids, hemostatics, cardiac glycosides, histamine H2 antagonists, hormones und their inhibitors, immunotherapeutics, cardiotonics, coccidiostatics, laxatives, lipid-lowering agents, gastrointestinal therapeutics, malaria therapeutics, migrane agents, microbicides, Crohn's disease, metastasis inhibitors, migrane agents, mineral preparations, motility-increasing active ingredients, muscle relaxants, neuroleptics, active ingredients for the treatment of estrogens, osteoporosis, otologicals, Parkinson agents, phytopharmaceuticals, pitavastatin, proton pump inhibitors, prostaglandins, active ingredients for the treatment of benign prostate hyperblasia, active ingredients for the treatment of pruritus, psoriasis active ingredients, psychopharmaceuticals, free radical scavengers, rennin antagonists, thyroid gland therapeutics, active ingredients for the treatment of seborrhea, active ingredients against seasickness, spasmolytics, alpha- und beta-sympathomimetics, tenatoprazole, platelet aggregation inhibitors, tyrosine kinase inhibitors, tranquillizers, ulcer therapeutics, further ulcer therapeutics, agents for the treatment of urolithiasis, virustatics, virustatics, vitamins, cytokines, active ingredients for combination therapy with cytostatics, cytostatics.

Active Ingredients

Examples of suitable active ingredients are acarbose, acetylsalicylic acid, abacavir, aceclofenac, aclarubicin, acyclovir, actinomycin, adalimumab, adefovir, adefovirdipivoxil, adenosylmethionine, adrenalin and adrenalin derivatives, agalsidase alpha, agalsidase beta, alemtuzumab, almotriptan, alphacept, allopurinol, almotriptan, alosetrone, alprostadil, amantadine, ambroxole, amisulpride, amlodipine, amoxicillin, 5-aminosalicylic acid, amitriptyline, amlodipine, amoxicillin, amprenavir, anagrelide, anakinra, anastrozole, androcen and androgen derivatives, apomorphine, aripiprazole, arsenic trioxide, artemether, atenolol, atorvastatin, atosiban, azathioprine, azelaic acid, barbituric acid derivatives, balsalazide, basiliximab, beclapermin, beclomethasone, bemiparin, benzodiazepines, beta-histine, bexaroten, bezafibrate, bicalutamide, bimatoprost, bosentan, botulinum toxim, brimonidine, brinzolamide, budesonide, budipine, bufexamac, bumetanide, buprenorphine, bupropion, butizine, calcitonin, calcium antagonists, calcium salts, candesartan, capecitabin, captopril, carbamazepine, carifenacin, carvedilol, caspofungin, cefaclor, cefadroxil, cefalexine, cefalosporins, cefditoren, cefprozil, cefuroxime, celecoxib, cepecitabin, cerivastatim, cetirizine, cetrorelix, cetuximab, chenodeoxycholic acid, choriogonadotropin, ciclosporin, cidofovir, cimetidine, ciprofloxacin, cisplatin, cladribin, clarithromycin, clavulanic acid, clindamycin, clobutinol, clonidine, clopidogrel, codeine, caffeine, colestyramine, cromoglicic acid, cotrimoxazole, coumarin and coumarin derivatives, darbepoetin, cysteamine, cysteine, cytarabine, cyclophosphamide, cyproterone, cytarabine, daclizumab, dalfopristin, danaparoid, dapiprazole, darbepoetin, defepripron, desipramine, desirudin, desloaratadin, desmopressin, desogestrel, desonide, dexibuprofen, dexketoprofen, disoproxil, diazepam and diazepam derivatives, didanosine, dihydralazine, diltiazem, dimenhydrinate, dimethyl sulfoxide, dimethicone, dipivoxil, dipyridarnol, dolasetron, domperidone and domperidan derivatives, donepzil, dopamine, doxazosine, doxorubicin, doxylamine, diclofenac, divalproex, dronabinol, drospirenone, drotrecogin alpha, dutasteride, ebastin, econazole, efavirenz, eletripan, emidastin, emtricitabin, enalapril, encepur, entacapon, enfurvirtide, ephedrine, epinephrine, eplerenone, epoetin and epoetin derivatives, eprosartan, eptifibatide, ertapenem, esomeprazole, estrogen and estrogen derivatives, etanercept, ethenzamide, ethynestradiol, etofenamate, etofibrate, etofylline, etonogestrel, etoposide, exemestan, ezetimib, famciclovir, famotidine, faropenandaloxate, felodipine, fenofibrate, fentanyl, fenticonazole, fexofenadine, finasteride, fluconazole fludarabine, flunarizine, fluorouracil, fluoxetin, flurbiprofen, flupirtin, flutamide, fluvastatin, follitropin, fomivirsen, fondaparinux, formoterol, fosfomicin, frovatriptan, furosemide, fusidic acid, gadobenate, galantamin, gallopamil, ganciclovir, ganirelix, gatifloxacin, gefitinib, gemfibrozil, gemopatrilate, gentamicin, gepirone, gestagen and gestagen derivatives, ginkgo, glatiramer, glibenclamide, glipizide, glucagon, glucitol and glucitol derivatives, glucosamine and glucosamine derivatives, glycoside antibiotics, glutathione, glycerol and glycerol derivatives, hypothalamus hormones, goserelin, grepafloxacin, gyrase inhibitors, guanethidine, gyrase inhibitors, hemin, halofantrin, haloperidol, urea derivatives as oral antidiabetics, heparin and heparin derivatives, cardiac glycosides, hyaluronic acid, hydralazine, hydrochlorothiazide and hydrochlorothiazide derivatives, hydroxyomeprazole, hydroxyzine, ibritumomab, ibuprofen, idarubicin, ifliximab, ifosfamide, iloprost, imatinib, imidapril, imiglucerase, imipramine, imiquimod, imidapril, indometacin, indoramine, infliximab, insulin, insulin glargin, interferons, irbesartan, irinotecan, isoconazole, isoprenaline, itraconazole, ivabradine, iodine and iodine derivatives, St John's wort, potassium salts, ketoconazole, ketoprofen, ketotifen, lacidipine, lamotrigin, lansoprazole, laronidase, latanoprost, leflunomide, leminoprazole, lepirudine, lercanidipine, leteprinim, letrozole, levacetylmethadol, levetiracetam, levocetirizine, levodopa, levodrpropicin, levofloxacin, levomethadone, licofelone, linezolide, lipinavir, lipoic acid and lipoic acid derivatives, lisinopril, lisuride, lofepramine, lodoxamide, lomefloxacin, lomustine, loperamide, lopinavir, loratadine, lornoxicam, losartan, lumefantrin, lutropine, magnesium salts, macrolide antibiotics, mangafodipir, maprotilin, mebendazole, mebeverine, meclozine, mefenamic acid, mefloquin, meloxicam, memantine, mepindolol, meprobamate, meropenem, mesalazine, mesuximide, metamizole, metformin, methadone, methotrexate, methyl 5-amino-4-oxopentanoate, methylnaloxone, methyl-naltrexone, methylphenidate, methylprednisolone, metixen, metoclopramide, metoprolol, metronidazole, mianserine, mibefradil, miconazole, mifepristone, miglitol, miglustad, milnacipran, minocycline, minoxidil, misoprostol, mitomycin, mizolastine, modafinil, moexipril, montelukast, moroctocog, morphinans, morphine and morphine derivatives, moxifloxacin, ergot alkaloids, nalbuphine, naloxone, naproxen, naratriptan, narcotine, natamycin, nateglinide, nebivolol nefazodone, nelfinavir, neostigmine, neramexan, nevirapin, nicergoline, nicethamide, nifedipine, niflumic acid, nimodipine, nimorazole, nimustine, nesiritide, nisoldipine, norfloxacin, novamin sulfone, noscapine, nystatin, ofloxacin, octotride, olanzapine, olmesartan, olsalazine, oseltamivir, omapatrilate, omeprazole, omoconazole, ondansetron, orlistate, oseltamivir, oxaceprol, oxacillin, oxaliplatin, oxaprozine, oxcarbacepin, oxicodone, oxiconazole, oxymetazoline, palivizumab, palonosetrone, pantoprazole, paracetamol, parecoxib, paroxetine, pegaspargase, peg-interferon, pegfilgrastrim, penciclovir, oral penicillins, pentazocine, pentifylline, pentoxifylline, peptide antibiotics, perindopril, perphenazine, pethidine, plant extracts, phenazone, pheniramine, phenylbutyric acid, phenytoin, phenothiazine, phenserine, phenylbutazone, phenytoin, pimecrolimus, pimozide, pindolol, pioglitazone, piperazine, piracetam, pirenzepine, piribedil, pirlindol, piroxicam, posaconazole, pramipexol, pramlintide, pravastatin, prazosine, procaine, promazine, propiverine, propranolol propionic acid derivatives, propyphenazone, prostaglandins, protionamide, proxyphylline, quetiapin, quinapril, quinaprilate, quinupristine, ramipril, ranitidine, rabeprazole, raloxifen, ranolazinie, rasburicase, reboxetin, repaclinide, reproterol, reserpine, revofloxacin, ribavirin, rifampicin, riluzole, rimexolone, risedronate, risperidone, ritonavir, rituximab, rivastimen, risatriptan, rofecoxib, ropinirol, ropivacaine, rosiglitazone, rotigotine, roxatidine, roxithromycin, ruscogenin, rosuvastatin, rutoside and rutoside derivatives, sabadilla, salbutamol, salicylate, salmeterol, saperconazole, thyroid gland hormones, scopolamine, selegilin, sertaconazole, sertindole, sertralin, sevelamer, sibutramine, sildenafil, silicates, simvastatin, sirolimus, sitosterin, sotalol, spaglumic acid, sparfloxacin, spectinomycin, spiramycin, spirapril, spironolactone, stavudine, streptomycin, sucralfate, sufentanil, sulbactam, sulfonamides, sulfasalazine, sulpiride, sultamicillin, sultiam, sumatriptan, suxamethonium chloride, tacrin, tacrolinmus, tadalafil, taliolol, talsaclidine, tamoxifen, tamsulosin, tasonermin, tazaroten, tegafur, tegaserod, telithromycin, telmisartan, temoporfin, temozolomide, tenatoprazole, tenecteplase, teniposide, tenofovir, tenoxicam, teriparatide, terazosine, terbinafin, terbutaline, terfenadine, teriparatide, terlipressin, tertatolol, testosterone and testosterone derivatives, tetracyclines, tetryzoline, tezosentan, theobromine, theophylline, theophylline derivatives, thiamazole, thiotepa, thr. growth factors, tiagabin, tiapride, tibolone, ticlopidine, tilidine, timolol, tinidazole, tioconazole, tioguanine, tiotropium, tioxolone, tirazetam, tiropramide, trofiban, tizanidine, tolazoline, tolbutamide, tolcapone, tolnaftate, tolperisone, tolterodine, topiramate, topotecan, torasemide, tramadol, tramazoline trandolapril tranylcypromine, trapidil, trastuzumab, travoprost, trazodore, trepostinil, triamcinolone and triamcinolone derivatives, triamterene, trifluperidol, trifluridine, trimetazidine, trimethoprim, trimipramine, tripelennamine, triprolidine, trifosfamide, tromantadine, trometamol, tropalpine, trovafloxacin, troxerutin, tulobuterol, trypsins, tyramine, tyrothricin, urapidil, ursodeoxycholic acid, theophylline ursodeoxycholic acid, valaciclovir, valdecoxib, valganciclovir, valproic acid, valsartan, vancomycin, vardenafil, vecuronium chloride, venlafaxin, verapamil, verteporfin, vidarabine, vigabatrin, viloxazine, vinblastine, vincamine, vincristine, vindesine, vinorelbin, vinpocetin, viquidil, vitamin D and derivatives of vitamin D, voriconazole, warfarin, xantinol nicotinate, ximelagatran, xipamide, zafirlucast, zalcitabin, zaleplon, zanamivir, zidovudine, ziprasidone, zoledronic acid, zolmitriptan, zolpidem, zoplicon, zotepine and the like.

The active ingredients can optionally also be used in the form of their pharmaceutically acceptable salts or derivatives, and in the case of chiral active ingredients both optically active isomers and racemates or diastereomer mixtures can be employed. Optionally, the pharmaceutical forms can also contain two or more pharmaceutical active ingredients.

### EXAMPLES

General experimental procedure for all examples:
Core material: 1000 g of theophylline granules 0.3 -0.8 mm

Spray suspensions:

| | | | Formulation |
|---|---|---|---|
| | Function | Material | amount [g] |
| 1. | Film former | EUDRAGIT ® RS 30 D | 300.0 |
| | | EUDRAGIT ® RL 30 D | 33.3 |
| 2. | Plasticizer | Triethyl citrate (TEC) | 20.0 |
| 3. | Release agent | Silicic acid (Syloid 244 FP) | 30.0 |
| 4. | Diluent | Purified water | 366.7 |
| | | Total: | 750.0 |
| | | Polymer content [% by weight] : | 13.3 |
| | | Solids content [% by weight] : | 20.0 |
| | | Polymer application [% by weight] | 10.0 |
| | | Total application [% by weight] | 15.0 |

Spray application:

| Apparatus: | GPCG 1.1 with top spray insert |
|---|---|
| Nozzle diameter: | 1.2 mm |
| Nozzle distance to the | 10 cm |
| product: | |
| Spray pressure: | 1.8 bar |
| Feed air amount [m³/h]: | 77-94 |
| Feed air temperature | 32-38 |
| [° C.]: | |
| Spray rate [g/min × kg]: | 6.0-8.2 |
| Spray period [min]: | 92-94 |
| Product temperature | 25.0-29.0 |
| [° C.]: | |
| Relative waste air | 23.5-34.9 |
| humidity [%]: | |

After the spray application, the mixture was dried for 5 min without in the apparatus with gentle vortexing. Subsequently, the stabilization step was carried out over a period of up to 2 hours with sampling after defined times. All samples were investigated with respect to active ingredient delivery rate. For the confirmation of the stabilization, the remaining amount of product was additionally poured onto racks according to the prior art and subsequently treated for 24 hours at 40° C. in a recirculating air drying cabinet (reference sample).

Determination of the Active Ingredient Release:

The determination of the active ingredient release was carried out analogously to USP27-NF22 <724> Drug Release, Extended Release, Apparatus 2.

Initially, the samples to be investigated were tested in 800 ml of simulated gastric juice for 2 h before they were rebuffered to pH 6.8 using 100 ml of phosphate buffer.

Medium 1:

Buffer pH 1.2 (simulated gastric juice)

| | |
|---|---|
| HCl, 1 mol/l | 1000 ml |
| Water | 9000 ml |

Medium 2:

Rebuffering to pH 6.8 by addition of

| | |
|---|---|
| Na₃PO₄ | 304 g |
| Water | 2000 ml |

Apparatus Parameters:

| | |
|---|---|
| Sample amount: | 300 mg |
| Analyses per sample: | n = 2 |
| Stirring speed | 150 rpm |
| Buffer volume: | 800 ml + 100 ml |
| Analysis period: | 8 hours |
| Wavelength: | 271 nm |

Stable Active Ingredient Release Profile

The present examples serve only for the illustration of the invention. A pharmaceutical form was fictitiously assumed which has a declared individual dose of 300 mg pellets where X=260 mg of active ingredient, here theophylline, =100% active ingredient and at the declared times 4 and 8 hours releases aliquot amounts T thereof. In addition, the measurements were also carried out at the times 5 min, 15 min, 30 min, 45 min, 1 h, 1.5 h, 2 h, 2.5 h, 3 h, 3.5, 5 h, 6 h and 7 h. For the sake of clarity the individual measurements are not listed. However, in the case of all “stable” pharmaceutical forms according to the invention, they were also within the defined tolerance at these times.

The success of the conditioning can be monitored by the comparison of the course of the curve of the conditioned pharmaceutical form according to the invention with the reference conditioning. Since this description would be unclear here owing to the large number of superimposed curves and individual measuring points, in a simplified manner one or more representative points are shown in tabular form.

### Example 1 (According to the Invention)

Aftertreatment by spraying in 240 g of water over 30 min using the following parameters:

| | |
|---|---|
| Spray rate [g/kg × min]: | 7.7-8.2 |
| Feed air temperature | 75.0 |
| [° C.]: | |
| Feed air amount [m³/h]: | 83-87 |
| Product temperature | 49.0-52.0 |
| [° C.]: | |
| Relative humidity in | 8.1%-10.1% |
| the waste air [%]: | |

Active ingredient release:

| | After spray | After 5 min | After 30 min | After 24 h |
|---|---|---|---|---|
| Measurement | application | afterdrying | stabilization | afterdrying |
| points [min] | [%] | [%] | [%] | [%] |
| 240 | 73.08 | 76.00 | 44.43 | 47.56 |
| 480 | 97.60 | 98.26 | 61.17 | 63.55 |

Assessment:

The aftertreatment leads to a slowing of the release of active ingredient and after 30 min to a stable active ingredient release profile.

### Example 2 (According to the Invention)

Aftertreatment by spraying in 487 g of water over 32 min using the following parameters:

| | |
|---|---|
| Spray rate [g/kg × min]: | 10.1-14.4 |
| Feed air temperauure | 80 |
| [° C.]: | |
| Feed air amount [m³/h]: | 74-76 |
| Product temperature | 47.0-49.0 |
| [° C.]: | |
| Relative humidity in | 11.8-16.9 |
| the waste air [%]: | |

Active Ingredient Release:

| | After spray | After 5 min | After 30 min | After 24 h |
|---|---|---|---|---|
| Measurement | application | afterdrying | stabilization | afterdrying |
| points [min] | [%] | [%] | [%] | [%] |
| 240 | 66.09 | 72.15 | 42.91 | 42.81 |
| 480 | 94.50 | 95.59 | 61.83 | 61.82 |

Assessment:

The aftertreatment leads after 30 min to a stable active ingredient release profile.

### Example 3 (According to the Invention)

Spray application according to formulation 1 and process A.

Aftertreatment by spraying in 950 g of water over 30 min using the following parameters:

| | |
|---|---|
| Spray rate [g/kg × min]: | 24.7-34.8 |
| [° C.]: | 86-90 |
| Feed air amount [m³/h]: | 81-88 |
| Product temperature | 42.0-45.0 |
| [° C.]: | |
| Relative humidity in | 19.7-29.6 |
| the waste air [%]: | |

Active Ingredient Release:

| | After spray | After 5 min | After 30 min | After 24 h |
|---|---|---|---|---|
| Measurement | application | afterdrying | stabilization | afterdrying |
| points [min] | [%] | [%] | [%] | [%] |
| 240 | 75.55 | 67.39 | 53.68 | 50.72 |
| 480 | 96.79 | 91.13 | 73.77 | 70.31 |

Assessment:

The aftertreatment leads to a slowing of the release of active ingredient. A stable active ingredient release profile is achieved after 30 min.

### Example 4 (According to the Invention)

Aftertreatment by spraying in 240 g of water over 30 min using the following parameters:

| | |
|---|---|
| Spray rate [g/kg × min]: | 8.5 |
| Feed air temperature | 75-80 |
| [° C.]: | |
| Feed air amount [m³/h]: | 71-88 |
| Product temperature | 47-48 |
| [° C.]: | |
| Relative humidity in | 8.9-10.2 |
| the waste air [%]: | |

Active Ingredient Release:

| | | After 5 | | | | |
|---|---|---|---|---|---|---|
| | | min | After 10 | After 20 | After 30 | After 24 h |
| Measurement | After spray | after- | min | min | min | after- |
| points | application | drying | stabiliz- | stabiliz- | stabiliz- | drying |
| [min] | [%] | [%] | ation [%] | ation [%] | ation [%] | [%] |
| 240 | 98.94 | 86.81 | 75.52 | 63.58 | 58.40 | 60.87 |
| 480 | 99.48 | 98.95 | 95.02 | 83.34 | 76.43 | 78.07 |

Assessment:

The aftertreatment leads to a monotonous slowing of the release of active ingredient. A stable active ingredient release profile is achieved after 20 min.

Long-term Storage Stability (Storage in Aluminum Cans):

For checking the behavior on relatively long storage, the pellets from example 4 stabilized for 30 min according to the invention are stored for 1 or 3 months in aluminum cans and subsequently tested again.

| | | After 1 month's storage | | | After 3 month's storage | | |
|---|---|---|---|---|---|---|---|
| | | 25° C., | 30° C., | 40° C., | 25° C., | 30° C., | 40° C., |
| Measuring | After 30 min | 60% rel. | 60% rel. | 75% rel. | 60% rel. | 60% rel. | 75% rel. |
| points | stabilization | humidity | humidity | humidity | humidity | humidity | humidity |
| [min] | [%] | [%] | [%] | [%] | [%] | [%] | [%] |
| 240 | 58.40 | 59.67 | 59.69 | 55.73 | 58.75 | 58.86 | 52.23 |
| 480 | 76.43 | 78.37 | 77.65 | 73.19 | 77.22 | 77.03 | 71.31 |

Result: The differences from the intended release profile are within the intended tolerance. The pharmaceutical form is thus to be regarded as stable.

### Example 5(According to the Invention)

Aftertreatment by spraying in 240 g of a 10% strength suspension of precipitated silicic acid (Syloid) in water over 30 min using the following parameters:

| | |
|---|---|
| Spray rate [g/kg × min]: | 8.2 |
| Feed air temperature | 75 |
| [° C.]: | |
| Feed air amount [m³/h]: | 71-78 |
| Product temperature | 42-46 |
| [° C.]: | |
| Relative humidity in | 9.2%-10.1% |
| the waste air [%]: | |

Active Ingredient Release:

| Measurement | After spray | After 5 min | After 30 min | After 24 h |
|---|---|---|---|---|
| points | application | afterdrying | stabilization | afterdrying |
| [min] | [%] | [%] | [%] | [%] |
| 240 | 42.46 | 56.46 | 35.83 | 35.59 |
| 480 | 65.69 | 82.70 | 52.06 | 51.82 |

Assessment:

The aftertreatment leads to a slowing of the release of active ingredient. A stable active ingredient release profile is achieved after 30 min.

### Example 6 (According to the Invention)

Spray application according to formulation 1 and process A.

Aftertreatment by spraying in 240 g of a 10% strength suspension of precipitated silicic acid (Syloid) in water over 30 min using the following parameters:

| | |
|---|---|
| Spray rate [g/kg × min]: | 8.5 |
| Feed air temperature | 75-80 |
| [° C.]: | |
| Feed air amount [m³/h]: | 75-82 |
| Product temperature | 47-49 |
| [° C.]: | |
| Relative humidity in | 9.8-10.1 |
| the waste air [%]: | |

Active Ingredient Release:

| | | After 5 | | | | |
|---|---|---|---|---|---|---|
| | | min | After 10 | After 20 | After 30 | After 24 h |
| Measurement | After spray | after- | min | min | min | after- |
| points | application | drying | stabiliz- | stabiliz- | stabiliz- | drying |
| [min] | [%] | [%] | ation [%] | ation [%] | ation [%] | [%] |
| 240 | 98.58 | 60.20 | 40.73 | 40.62 | 36.67 | 38.9 |
| 480 | 99.55 | 86.90 | 60.17 | 58.33 | 55.08 | 56.6 |

Assessment:

The aftertreatment leads to a monotonous slowing of the release of active ingredient. A stable active ingredient release profile is achieved after 10 min. Long-term storage stability (storage in aluminum cans): For checking the behavior on relatively long storage, the pellets from example 6 stabilized for 30 min according to the invention are stored for 1 or 3 months in aluminum cans and subsequently tested again.

| | | After 1 month's storage | | | After 3 month's storage | | |
|---|---|---|---|---|---|---|---|
| | | 25° C., | 30° C., | 40° C., | 25° C., | 30° C., | 40° C., |
| Measuring | After 30 min | 60% rel. | 60% rel. | 75% rel. | 60% rel. | 60% rel. | 75% rel. |
| points | stabilization | humidity | humidity | humidity | humidity | humidity | humidity |
| [min] | [%] | [%] | [%] | [%] | [%] | [%] | [%] |
| 240 | 36.67 | 40.59 | 40.09 | 31.50 | 35.46 | 35.88 | 28.64 |
| 480 | 55.08 | 61.82 | 59.21 | 48.48 | 54.20 | 53.79 | 46.39 |

Result: The differences from the intended release profile are within the intended tolerance. The pharmaceutical form is thus to be regarded as stable.

### Example 7 (Conditioning Temperature not According to the Invention)

In order to demonstrate the influence of the temperature of the conditioning temperature (corresponding to the product temperature) and of the atmospheric humidity relative humidity in the waste air), both are set outside the range according to the invention.

Aftertreatment by spraying in 240 g of a 10% strength suspension of precipitated silicic acid Syloid) in water over 30 min using the following parameters:

| | |
|---|---|
| Spray rate [g/kg × min]: | 8.5 |
| Feed air temperature | 30-33 |
| [° C.]: | |
| Feed air amount [m³/h]: | 84-83 |
| Product temperature | 24-26 |
| [° C.]: | |
| Relative huaiidity in | 36.9-40.2 |
| the waste air [%]: | |

Active Ingredient Release:

| | | After 5 | | | | |
|---|---|---|---|---|---|---|
| | | min | After 10 | After 20 | After 30 | After 24 h |
| Measurement | After spray | after- | min | min | min | after- |
| points | application | drying | stabiliz- | stabiliz- | stabiliz- | drying |
| [min] | [%] | [%] | ation [%] | ation [%] | ation [%] | [%] |
| 240 | 98.04 | 97.91 | 98.95 | 98.73 | 98.87 | 92.75 |
| 480 | 99.07 | 99.00 | 99.18 | 98.92 | 99.30 | 99.07 |

Assessment:

The aftertreatment does not lead to a retarded active ingredient release profile after 30 min. The permeability of the coating, with a release of about 100% after 4 or 8 hours, is very high and indicates damage as a result of agglutination in too great a humidity.

1. A method for producing pharmaceutical forms having a stable active ingredient release profile, the pharmaceutical forms having controlled release characteristics on account of a coating of vinyl (co)polymers,
characterized in that
the coated pharmaceutical forms are conditioned at a temperature of 30 to 70° C. in a fluidized bed coating apparatus or a drum coating apparatus for at least 10 minutes until achievement of a stable active ingredient release profile, an atmospheric humidity of 5 to 30% being set during the conditioning.

2. The method as claimed in claim 1, characterized in that the coated pharmaceutical forms are produced in a first production step by coating active ingredient-containing cores or pellet cores with vinyl (co)polymers in a fluidized bed coating apparatus or a drum coating apparatus and optionally drying and in a second step carrying out the conditioning of the coated pharmaceutical forms in the same apparatus immediately after their production.

3. The method as claimed in claim 1, characterized in that the necessary atmospheric humidity is adjusted by spraying in water.

4. The method as claimed in claim 1, characterized in that an aqueous suspension comprising 0.1 to 5% by weight of release agent based on the suspension, is sprayed in.

5. The method as claimed in claim 4, characterized in that the release agent employed is talc, magnesium stearate or a silicic acid.

6. The method as claimed in claim 1, characterized in that the coatings of the pharmaceutical forms consist of (meth)acrylate (co)polymers or of polyvinyl acetate or derivatives of polyvinyl acetate.

7. The method as claimed in claim 6, characterized in that the coatings of the pharmaceutical forms consist of (meth)acrylate (co)polymers which are polymerized to more than 95% by weight to 100% from monomers containing neutral radicals.

8. The method as claimed in claim 7, characterized in that the (meth)acrylate copolymers are polymerized from 20 to 40% by weight of ethyl acrylate and 60 to 80% by weight of methyl methacrylate.

9. The method as claimed in claim 6, characterized in that the coatings of the pharmaceutical forms consist of cationic (meth)acrylate copolymers.

10. The method as claimed in claim 9, characterized in that the coatings of the pharmaceutical forms consist of (meth)acrylate copolymers which contain quaternary amino groups.

11. The method as claimed in claim 10, characterized in that the coating of the pharmaceutical forms consist of (meth)acrylate copolymers which are synthesized from free radical-polymerized units of 50-70% by weight of methyl methacrylate, 20-40% by weight of ethyl acrylate and 12-2% by weight of 2-trimethylammonium ethyl methacrylate chloride.

12. The method as claimed in claim 9, characterized in that the coatings of the pharmaceutical forms consist of (meth)acrylate copolymers which contain tertiary amino groups.

13. The method as claimed in claim 12, characterized in that the coatings of the pharmaceutical forms consist of (meth)acrylate copolymers which are synthesized from 20-30% by weight of methyl methacrylate, 20-30% by weight of butyl methacrylate and 60-40% by weight of dimethylaminoethyl methacrylate.

14. The method as claimed in claim 6, characterized in that the coatings of the pharmaceutical forms consist of anionic (meth)acrylate (co)polymers.

15. The method as claimed in claim 4, characterized in that the coatings of the pharmaceutical forms consist of anionic (meth)acrylate (co)polymers which consist to 25 to 95% by weight of free radical-polymerized C₁- to C₄-alkyl esters of acrylic or methacrylic acid and to 5 to 75% by weight of (meth)acrylate monomers containing an anionic group in the alkyl radical.
